(19) United States
(12) Patent Application Publication
(10) Pub. No.: US 2007/0142321 A1
(43) Pub. Date: Jun. 21, 2007

(54) METHOD FOR PREVENTING GROWTH OF BACTERIA ON CONTACT LENSES WITH EYE DROPS

(76) Inventors: Roya Borazjani, Ft. Worth, TX (US); Joseph C. Salamone, Boca Raton, FL (US); Daniel Ammon JR., Penfield, NY (US)

Correspondence Address: Bausch & Lomb Incorporated, One Bausch & Lomb Place, Rochester, NY, 14604-2701, US

(21) Appl. No.: 11/611,328

(22) Filed: Dec. 15, 2006

Related U.S. Application Data
(60) Provisional application No. 60/752,427, filed on Dec. 21, 2005.

(51) Int. Cl. A61K 31/715; A61K 31/155; A61K 31/44; A61K 31/14

(52) U.S. Cl. 514/54; 514/635; 514/358; 514/642

(57) ABSTRACT

The present invention relates to a novel eye-drop solution that has superior microbe and bacteria management properties. Regular use in the eye will prevent bacteria from being attached to the eye. Regular use of the eye-drops will also prevent growth of microbes in the eye. A patient that uses this invention can slow the progression of infection and growth of microbes between cleaning. For disposable contact lenses the antimicrobial management properties reduces the risk of infection during the extended wear period. In one embodiment, there is a method of treating contact lenses comprising administering to the lens an ophthalmically safe composition comprising an amount of antimicrobial agent that prevents growth of microbes in the eye but is a less than the minimum disinfecting amount.

### CROSS REFERENCE

This application claims the benefit of Provisional Patent Application No. 60/752,427 filed Dec. 21, 2005 and is incorporated herein by reference.

### BACKGROUND OF THE INVENTION

1. Field of the Invention

This invention relates to the management of microbes on contact lenses with eye-drops and more particularly to the management of microbes on contact lenses that are worn for longer than one-day without removal with eye-drops.

2. Discussion of the Related Art

Contact lenses in wide use today fall into two general categories, hard and soft. The hard or rigid corneal type lenses are formed from materials prepared by the polymerization of acrylic esters, such as poly(methyl methacrylate) (PMMA). The gel, hydrogel or soft type lenses are made by polymerizing such monomers as 2-hydroxyethyl methacrylate (HEMA) or, in the case of extended wear lenses, by polymerizing silicon-containing monomers or macromonomers. Silicone-containing monomers and macromonomers are referred to as silicone hydrogel contact lenses. The silicone hydrogel contact lenses have a high oxygen permeability that allow the eye tissue to absorb oxygen at a rate that keeps tissue healthy. Thus, silicone hydrogels or other high oxygen permeable materials can be worn for extended periods of time and are often referred to as extended wear contact lenses. By extended wear contact lenses, it is meant contact lenses that are worn for longer than a day without removal from the eye.

One challenge of extended wear contact lenses is keeping the lenses free of microbes, protein deposits, lipid deposits and debris. Extended wearers of contact lenses, optionally, use multipurpose cleaning solutions for cleaning contact lenses when the contact lenses are removed from the patient's eyes. The amount of disinfecting agent in a multipurpose solution will disinfect contact lenses according to the regimens recommended for patient use. Multipurpose contact lens cleaning solutions include Optifree® Express by Alcon Laboratories, Forth Worth, Tex.; ReNu® with MoistureLoc by Bausch & Lomb, Incorporated, Rochester, N.Y.; ReNu® Multiplus by Bausch & Lomb, Incorporated, Rochester, N.Y.; Complete® Moisture Plus by Advanced Medical Optics, Santa Ana, Calif. U.S. Patent Publication No. 2005-0202986 discloses a multipurpose cleaning solution with poloxamine 407 and poloxamer 1304.

Eye-drop solution for cleaning contact lenses are formulated to clean while the lens is placed in the eye. Eye-drops include but are not limited to Complete® Blink & Clean by Advanced Medical Optics, Santa Ana, Calif. and ReNu® MultiPlus Lubricating and Rewetting Drops, Bausch & Lomb Incorporated, Rochester, N.Y. Presently, the amount of antimicrobial agent in eye-drops is the distinguishing feature between some multipurpose solutions and some related eyedrops. Eyedrops are prepared with enough antimicrobial agent to preserve the solution. However, there has yet to be developed an eye-drop solution that has in-eye antimicrobial properties. When instilled in the eye, the preservative amount of the antimicrobial agent is diluted by the lacrimal fluid and is rapidly removed from the eye. Within five minutes, the effective amount of an eyedrop, typically, is washed from the eye. It would be of great advantage to have an eye-drop solution that has the ability to manage microbes that is in the eye by preventing microbial growth and preventing microbial attachment in the eye.

Despite the availability of various contact lens disinfecting systems such as those discussed above, there continues to be a need for improved disinfecting systems that can manage antimicrobial agents when instilled in the eye of a patient, but still be gentle enough not to harm a patient. Such improved disinfecting systems are beneficial if they are simple to use, are effective against a broad spectrum of microbes, are non-toxic and do not cause ocular irritation as the result of binding to the contact lens material. There is a particular need in the field of contact lens disinfection and ophthalmic composition preservation for safe and effective chemical agents with antimicrobial activity. The present invention addresses these and other needs.

### BRIEF DESCRIPTION OF THE DRAWINGS

FIG. 1 is a chart showing the results of the Bioburden Reduction Assay for S. aureus on PureVision® contact lenses for Formula 1, Formula 2, Formula 3, Borate and PBS.

FIG. 2 is a chart showing the results of the Bioburden Reduction Assay for P. aeruginosa on PureVision® contact lenses for Formula 1, Formula 2, Formula 3, a borate solution and phosphate buffered saline solution.

FIG. 3 is a chart showing the results of the Bioburden Reduction Assay for S. aureus on PureVision® contact lenses for Formula 1, Formula 2, Formula 3, a borate solution and phosphate buffered saline solution.

### SUMMARY OF THE INVENTION

The present invention relates to a novel eye-drop solution that has superior bacteria management properties. Regular use in the eye will prevent bacteria from being attached to the eye. Regular use of the eye-drops will also prevent growth of bacteria in the eye. A patient that uses this invention can slow the progression of infection and growth of bacteria between cleaning where bacteria can be destroyed and removed. For disposable contact lenses the antimicrobial management properties reduces the risk of infection during the extended wear period. In one embodiment, there is a method of treating contact lenses comprising administering to the lens an ophthalmically safe composition comprising an amount of antimicrobial agent that prevents growth of one or more microbes in the eye but is a less than the minimum disinfecting amount.

In another embodiment, there is a method of treating contact lenses. The method comprises administering to the lens an ophthalmically safe composition. The composition comprises an antimicrobial agent in an amount that prevents attachment of bacteria in the eye but is a less than disinfecting amount.

In another embodiment, there is a method of treating contact lenses comprising the step of administering to the lens an ophthalmically safe composition. The composition comprises an antimicrobial agent in an amount that prevents growth of bacteria in the eye but is a less than the minimum disinfecting amount.

In one embodiment, the amount of antimicrobial agent is greater than the minimum preserving amount by a multiple of 1.5. Typically, the amount of antimicrobial agent in the ophthalmically safe composition is greater than the minimum preserving amount by a multiple of about 1.8, about 2.0, about 2.5, about 3.0.

In another embodiment, the antimicrobial agent is a quaternium containing antimicrobial agent. Preferably, according to one aspect of the invention, the antimicrobial agent is a biguanide containing antimicrobial agent. The antimicrobial agent is selected from the group consisting of poly(hexamethylene biguanide); polyquaternium-1, chlorhexadine, Alexidine and mixtures thereof. Preferably, the antimicrobial agent is Alexidine.

In one embodiment, the surfactant in the eye-drop solution is selected from the group consisting of poly(ethylene oxide)-poly(propylene oxide) block copolymers and combinations thereof. Typically, the amount of surfactant in the eye-drop solution is a minimum of about 0.5 wt. % and a maximum of about 10 wt %.

In one embodiment, the eye-drop solution comprises a cationic polysaccharide. The amount of cationic polysaccharides in the eye-drop solution is a minimum of about 0.001 wt. % and a maximum of about 0.5 wt. %.

In one embodiment, the amount of antimicrobial agent is greater than the minimum preserving amount by a multiple of 1.5. Typically, the amount of antimicrobial agent in the ophthalmically safe composition is greater than the minimum preserving amount by a multiple of about 1.8, about 2.0, about 2.5 or about 3.0.

Other advantages and features will be apparent from the below detailed description of the invention.

### DETAILED DESCRIPTION OF THE INVENTION

This invention is directed to an eye-drop composition that is effective at preventing growth of microbes in the eye of a patient and/or to prevent attachment of bacteria to a contact lens while worn in the eye of a patient. Preferably, in one embodiment, the present invention is directed to aqueous eye-drop compositions for cleaning lipid deposits and/or prevention of lipid deposition on medical devices, especially on contact lenses, and methods of using these compositions. The term “cleaning lipid deposits” includes preventing, removing and/or reducing the formation of lipid deposits. Combinations according to the invention have been found to improve the lipid cleaning properties for contact lenses and prevent the growth of microbes without adversely affecting the comfort or safety in terms of the level of toxicity to eye tissue.

Compositions of the present invention in solution are physiologically compatible or “ophthalmically safe” for use with contact lenses. “Ophthalmically safe” as used herein means that a contact lens treated with or in the subject solution is generally suitable and safe for direct placement on the eye without rinsing.

The subject solutions are safe and comfortable for daily contact with the eye via a contact lens that has been wetted with the solution. An ophthalmically safe solution typically has a tonicity and pH that is compatible with the eye and comprises materials, and amounts thereof, that are non-cytotoxic according to ISO (International Standards Organization) standards and U.S. FDA (Food and Drug Administration) regulations. The solutions should be sterile in that the absence of microbial contaminants in the product prior to release should be statistically demonstrated to the degree necessary for such products. In one embodiment the eye-drops of one or more embodiments are ophthalmic solutions or ophthalmically safe solutions.

Method of Using the Multipurpose Solution and the Eye-Drop Solution

Aqueous compositions of the invention are utilized as eye-drop solutions for contact lens care. Especially useful is the ability to clean contact lens and manage antimicrobial agents while the contact lenses are worn in the eye. Thus, as mentioned above, aqueous compositions according to the invention are especially advantageous with people who wear lenses under an extended-wear or continuous-wear regime. Extended wear is defined as a lens that is worn overnight, during sleep, preferably capable of wear for a week or more. Continuous wear is defined as a lens that is worn for at least 1 month. Eye-drop solutions according to the invention may suitably be applied as follows. During wear, about one or two drops are placed directly onto each lens whenever needed. Thereafter, the wearer should blink several times. It is also possible to use a spray mist to deliver the formulation to the eye.

Eye-Drop Solution

In one embodiment, the eye-drop solution contains an amount of antimicrobial agent that prevents growth of microbes in the eye but is a less than the minimum disinfecting amount.

For the purposes of this patent application, an amount of antimicrobial agent that prevents growth of microbes is defined as a concentration level of an antimicrobial agent in a test solution that prevents growth of microbes when diluted by a 0.9 wt. % saline solution in a 1:1 ratio of test solution to saline solution.

For the purposes of this patent application a disinfecting amount is an amount of an antimicrobial agent that passes the Stand Alone Disinfecting Efficacy Test by the Food and Drug Administration (510k) of the United States of America in a minimum of three out of four test samples in a test solution that is diluted with 0.9 wt. % saline solution in a 1:1 ratio of test solution to saline solution.

The FDA (510 (k)) Guidance Document for contact lens products defines procedures that measure the extend of viability loss of representative microorganisms at established time intervals to determine the extend of viability loss. FDA (510 (k)) Guidance Documents recommended test organisms for both disinfecting stand-alone and preservative efficacy testing are composed of three bacteria (Pseudomonas aeruginosa ATCC 9027, Stapylococcus aureus ATCC 6538, and Serratia marcescens ATCC 13880) and two fungi (Candida albicans ATCC 10231, and Fusarium solani ATCC 36031). The performance requirement biocidal stand alone testing calls for 3 log reduction for bacterial cells and 1 log reduction for each fungi. At day 28, after the rechallenge on day 14, the performance requirement for preservative efficacy testing calls for reduction of 3.0 logs per bacteria and ±0.5 for fungi.

For the purpose of this patent application an amount that prevents adhesion (or attachment) of bacteria in the eye is an amount that will result in a 50% reduction of adhesion of Pseudomonas aeruginosa GSU#3 to a contact lens according to the Bacterial Adhesion Test compared to a control lens of the same composition treated with a phosphate buffered saline solution.

“Bacterial Adhesion Test” means testing according to the following procedure with reference to Sawant, et al. “Radioopacity additives in silicone stent materials reduce in vitro bacterial adherence,” Curr. Micorbiol. 22:285-292 (1991) and Gabriel, et al., “Effects of sliver on adherence of bacteria to urinary catheter: in vitro studies,” Curr. Microbio., 30:17-22 (1995). Bacterial cells are selected and grown in Triptic Soy Broth (TSB) at 37° C. on a rotary shaker for 12 h to 18 h. The cells are harvested by centrifugation at 3000×g for 10 mm. Then, the cells are washed two times in 0.9% saline and suspended in minimal medium (1.0 g D-glucose, 7.0 g K₂HPO₄, 2.0 g KH₂PO₄, 0.5 g sodium citrate, 1.0 g (NH₄)SO₄, and 0.1 g MgSO₄ in 1 liter distilled H₂O, pH 7.2) to a concentration of about 2×10⁸ cells per ml (Optical density 0.10 at 600 nm). The minimal broth cultures are incubated for 1 h at 37° C. with shaking. One to 3˜tCi/ml of L-[3,4,5-³H]leucine (NEN Research Products, Du Pont Company, Wilmington, Del.) is added to the cells and the cell suspensions are incubated for another 20 mm. These cells are washed 4 times in 0.9% saline and suspended in phosphate buffered saline (PBS) to a concentration of about 108 cells per ml (Optical density 0.10 at 600 nm).

The contact lens is incubated with 3 ml of the radiolabeled cell suspension at 37° C. for 2 h. Then, lenses are removed from the cell suspension with a sterile forceps and immersed 5 times in each of three successive changes (180 ml) of initially sterile 0.9% saline. Then the lenses are shaken to remove the saline and transfer the lens to 20-ml glass scintillation vials. Ten milliliters of Opti-Fluor scintillation cocktail (Packard Instrument Co., Downers Grove, Ill.) are added to each vial. The vials are vortexed and then placed in a liquid scintillation counter (LS-7500, Beckman Instruments, Inc., Fullerton, Calif.).

Data for two separate samples are converted from disintegrations per milliliter (dpm) to colony-forming units (cfu) based on a standard calibration curve and expressed as cfu/mm². Calibration curves are constructed from numbers of colonies recovered in pour plates of serial dilutions of inocula and from optical densities (O.D.s) of serial dilutions of cell suspensions of known densities. Uninoculated contact lenses, which serve as controls for the nonspecific uptake of leucine, are treated in the same manner as the inoculated sections.

In yet another embodiment, the antimicrobial agent that is present in the eye-drop solution is selected from the group consisting of quaternary ammonium containing preservatives and combinations thereof. Typically, the antimicrobial agent is selected from the group consisting of biguanide containing preservatives and combinations thereof. In one embodiment, the amount of antimicrobial agent in the eye-drop solution is a minimum of about 1 ppm and a maximum of about 100 ppm but sufficient to prevent bacterial adhension or prevent cell growth as determined by routine experimental procedure. Typically, the amount of antimicrobial agent in the eye-drop solution is a minimum of about 2 ppm, about 3 ppm, about 4 ppm or about 5 ppm. Typically, the amount of antimicrobial agent in the eye-drop solution is a maximum of about 70 ppm, about 50 ppm, about 30 ppm, about 20 ppm, about 10 ppm or about 5 ppm.

Suitable antimicrobial agents for use in the eye-drop solution include, for example, but are not limited to 1,1′-hexamethylenebis[5-(p-chlorophenyl)biguanide] (Chlorhexidine) or water soluble salts thereof, 1,1′-hexamethylenebis[5-(2-ethylhexyl)biguanide] (Alexidine) or water soluble salts thereof, poly(hexamethylene biguanide) (PHMB) or water soluble salts thereof, polyquaternium-1 and quaternary ammonium esters. Biguanides are described in U.S. Pat. Nos. 5,990,174; 4,758,595 and 3,428,576, each incorporated herein in its entirety by reference. The preferred antimicrobial agents are poly(aminopropyl biguanide) (PAPB), also commonly referred to as poly(hexamethylene biguanide) (PHMB), and most preferably, the antimicrobial agents is 1,1′-hexamethylenebis[5-(2-ethylhexyl)biguanide] (Alexidine).

The eye-drop solutions according to the invention, typically, function to clean lipid deposits and/or prevent of lipid deposition on a medical device advantageously contain a surfactant. In one embodiment, the amount of surfactant in the eye-drop solution is a minimum of about 0.1 wt. % and a maximum of about 10 wt. %. Typically the amount of surfactant in the eye-drop solution is a minimum of about 0.5 wt. %, about 1 wt. %, about 1.5 wt. %, about 2 wt. %, about 2.5 wt. % or about 3 wt. % and/or a maximum of about 5 wt. %, about 4.5 wt. %, about 4 wt. %, about 3.5 wt. % or about 3 wt. %.

Poloxamers are block copolymers consisting of propylene oxide (PO) and ethylene oxide (EO) blocks—specifically, they are poly(oxyethylene-oxypropylene-oxyethylene) triblock copolymers. Their solubility in water is generally good, but the properties of the individual block copolymers vary substantially. The nomenclature used for the block copolymers, and generally herein, is such that the first two figures, when multiplied by 100, represent the average molecular weight of the PO block, whilst the last figure, when multiplied by 10, represents the ethylene oxide content (% w/w) of the poloxamer. Thus, for Pluronic F127 (poloxamine 407), the average molecular weight of the PO block is 12000 Daltons with 70% w/w ethylene oxide content. The straight chain polyether surfactants known as poloxamer surfactants are available from BASF Wyandotte Corp., Wyandotte, Mich., under the registered trademark “Pluronic™ (BASF).” For convenience purposes, the straight chain surfactants employed in the aqueous composition disclosed herein will be referred to as poloxamer and are generally with a numerical suffix to identify a particular grade of material.

In one embodiment, the surfactant in the eye-drop solution is selected from the group consisting of poloxamer 407, poloxamer 403, poloxamer 335 and poloxamine 1304.

Grades of poloxamine surfactants available with molecular weights ranging from as low as 1650 to 27,000. Properties of each grade within the series vary depending on the percent of hydrophilic units poly(oxyethylene) and molecular weight of hydrophobic units poly(oxypropylene) in the adduct. While all members within the series exhibit wetting and detergency properties, it was discovered that only certain members are suitable for use in the eye-drop solutions disclosed herein, due to the wide variation in performance characteristics regulated by their hydrophilic-hydrophobic balance. The poloxamine surfactants found suitable are those capable of demonstrating maximum cleaning efficiency in dispersing both protein and lipid deposits at ambient and elevated temperatures at lowest solution concentration without trade-offs in lens compatibility and toxicity levels, i.e. maintaining the lowest potential as an irritant to eye tissues.

The eye-drop solution of one embodiment of the present invention include but are not limited to
Pluronic F38™ (BASF) having a HLB of 31 and average molecular weight (AMW) of 4700;
Pluronic F68™ (BASF) having a HLB of 29 and AMW of 8400;
Pluronic 68LF™ (BASF) having a HLB of 26 and AMW or 7700;
Pluronic F77™ (BASF) having a HLB of 25 and AMW of 6600;
Pluronic F87™ (BASF) having a HLB of 24 and AMW of 7700;
Pluronic F88™ (BASF) having a HLB of 28 and AMW or 11400;
Pluronic F98™ (BASF) having a HLB of 28 and AMW of 13000;
Pluronic P105™ (BASF) having a HLB of 15 and AMW of 6500;
Pluronic F108™ (BASF) having a HLB of 27 and AMW of 14600;
Pluronic F127™ (BASF) having a HLB of 22 and AMW of 12600;
Pluronic L35™ (BASF) having a HLB of 19 and AMW of 1900;
Pluronic L42™ (BASF) having a HLB of 8 and AMW of 1630;
Pluronic L63™ (BASF) having a HLB of 11 and AMW of 2650;
Pluronic L101™ (BASF) having a HLB of 1 and AMW of 3800;
Pluronic P103™ (BASF) having a HLB of 9 and AMW of 4950;
Pluronic P123™ (BASF) having a HLB of 8 and AMW of 5750;
Pluronic L122™ (BASF) having a HLB of 4 and AMW of 5000;
Pluronic L121™ (BASF) having a HLB of 1 and AMW of 4400;
Pluronic L92™ (BASF) having a HLB of 6 and AMW of 3650;
Pluronic L81™ (BASF) having a HLB of 2 and AMW of 2750;
Pluronic L72™ (BASF) having a HLB of 7 and AMW of 2750;
Pluronic L62™ (BASF) having a HLB of 7 and AMW of 2500;
Pluronic L61 ™ (BASF) having a HLB of 3 and AMW of 2000;
Pluronic L31™ (BASF) having a HLB of 5 and AMW of 1100;
Pluronic F38™ (BASF) having a HLB of 31 and AMW of 4700;
Pluronic F68™ (BASF) having a HLB of 29 and AMW of 8400;
Pluronic 68LF™ (BASF) having a HLB of 26 and AMW of 7700;
Pluronic F77™ (BASF) having a HLB of 25 and AMW of 6600;
Pluronic F87™ (BASF) having a HLB of 24 and AMW of 7700;
Pluronic F88™ (BASF) having a HLB of 28 and AMW of 11400;
Pluronic F98™ (BASF) having a HLB of 28 and AMW of 13000;
Pluronic F108™ (BASF) having a HLB of 27 and AMW of 14600;
Pluronic F127™ (BASF) having a HLB of 22 and AMW of 12600;
Pluronic L35™ (BASF) having a HLB of 19 and AMW of 1900;
Tetronic 707™ (BASF) having a HLB of 27 and AMW of 12200;
Tetronic 908™ (BASF) having a HLB of 31 and AMW of 25000;
Tetronic 909™ (BASF) having a HLB of 32 and AMW of 30000;
Tetronic 1107™ (BASF) having a HLB of 24 and AMW of 15000;
Tetronic 1307™ (BASF) having a HLB of 24 and AMW of 18000; or
Tetronic 1508™ (BASF) having a HLB of 27 and AMW of 30000.

Another class of surfactants is the various polyethylene glycol ethers of stearyl alcohol. A specific example is steareth-100, available under the tradename Brij® 700 (ICI Americas).

Other non-ionic surfactants include: polyethylene glycol esters of fatty acids, e.g. coconut, castor oil, polysorbate, polyoxyethylene or polyoxypropylene ethers of higher alkanes (C₁₂-C₁₈); polysorbate 20 (available under the trademark Tween® 20); polyoxyethylene (23) lauryl ether (available under the tradename Brij® 35); polyoxyethyeneglycol (40) stearate (available under the tradename Myrj® 52); polyoxyethyeneglycol (20) stearate (available under the tradename Myrj® 49); and polyoxyethylene (25) propylene glycol stearate (available under the tradename Atlas® G 2612). Another surfactant that is useful in cleaning contact lenses is tyloxapol.

Various other surfactants suitable for use in the invention are disclosed in McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

The eye-drop solution of the present invention, optionally, comprises one or more cationic polysaccharides. Suitable cationic polysaccharides for use in compositions of the present invention include, for example, but are not limited to variations of polyquaternium-10 such as for example Polymer JR 125™ (Dow Chemical Company, Midland, Mich.) having a 2 percent solution viscosity of 75-125 cps and 1.5 to 2.2 percent nitrogen, Polymer JR 400™ (Dow Chemical Company) having a 2 percent solution viscosity of 300 to 500 cps and 1.5 to 2.2 percent nitrogen, Polymer JR 30M™ (Dow Chemical Company) having a 1 percent solution viscosity of 1,000 to 2,500 cps and 1.5 to 2.2 percent nitrogen, Polymer LR 400™ (Dow Chemical Company) having a 2 percent solution viscosity of 300 to 500 cps and 0.8 to 1.1 percent nitrogen, Polymer LR 30M™ (Dow Chemical Company) having a 1 percent solution viscosity of 1,250 to 2,250 cps and 0.8 to 1.1 percent nitrogen, and Polymer LK™ (Dow Chemical Company) having a 2 percent solution viscosity of 300 to 500 cps and 0.8 to 1.1 percent nitrogen. The preferred cationic polysaccharide for use in the present invention is Polymer JR 125™ or Polymer JR 400™.

In another embodiment, the amount of cationic polysaccharides in the eye-drop solution is a minimum of about 0.001 wt. % and a maximum of about 0.5 wt. %. Preferably, the amount of cationic polysaccharide in the eye-drop solution is a minimum of about 0.005 wt. %, about 0.01 wt. %, about 0.02 wt. %, about 0.04 wt. % and/or a maximum of about 0.05 wt. %, about 0.03 wt. %, about 0.01 wt. % or about 0.005 wt. %.

The eye-drop solutions of the present invention, typically, have an ophthalmically compatible pH, which generally will be a minimum of about 6, about 6.5 or about 7 and a maximum of about 8, about 7.8 or about 7.5. One or more conventional buffers may be employed to obtain the desired pH value. Suitable buffers include, for example, but are not limited to borate buffers based on boric acid and/or sodium borate, phosphate buffers based on Na₂HPO₄, NaH₂PO₄ and/or KH₂PO₄, citrate buffers based on sodium or potassium citrate and/or citric acid, sodium bicarbonate, amino acid buffers, aminoalcohol buffers, Good buffers and combinations thereof. Generally, buffers will be used in the eye-drop solution in amounts having a minimum of about 0.05 or about 0.1 and a maximum of about 2.5 weight percent or about 1.5 weight percent.

The eye-drop solution of the present invention, typically, include one or more tonicity agents to approximate the osmotic pressure of normal lachrymal fluids, which is equivalent to a 0.9 percent solution of sodium chloride or 2.5 percent glycerin solution. Examples of suitable tonicity agents include but are not limited to sodium and potassium chloride, dextrose, mannose, glycerin, calcium and magnesium chloride. These agents are typically used individually in amounts that are a minimum of about 0.01 wt. % or about 0.2 wt. % and/or a maximum of about 2.5 wt. % or 1.5 wt. %.

Preferably, the tonicity agent is employed in the eye-drop solutions in an amount to provide a final osmotic value that is a minimum of about 200 mOsm/kg, about 220 mOsm/kg and/or a maximum of about 450 mOsm/kg, about 350 mOsm/kg or about 320 mOsm/kg.

Eye-drop solutions of the present invention may likewise include a wetting agent, to facilitate the composition wetting the surface of a contact lens. Within the art, the term “humectant” is also commonly used to describe these materials. A first class of wetting agents are polymer wetting agents. Examples of suitable wetting agents include, for example, but are not limited to poly(vinyl alcohol) (PVA), poly(N-vinylpyrrolidone) (PVP), cellulose derivatives, guar and guar derivatives, and poly(ethylene glycol). Cellulose derivatives, guar and guar derivatives, and PVA may be used to also increase viscosity of the composition, and offer this advantage if desired. Specific cellulose derivatives include, for example, but are not limited to hydroxypropylmethylcellulose, carboxymethylcellulose, methylcellulose, hydroxyethylcellulose, and cationic cellulose derivatives. As disclosed in U.S. Pat. No. 6,274,133, cationic cellulosic polymers also help prevent accumulation of lipids and proteins on a hydrophilic lens surface. Such cationic cellulosic polymers include, for example, but are not limited to water soluble polymers commercially available under the CTFA (Cosmetic, Toiletry, and Fragrance Association) designation Polyquaternium-10, including the cationic cellulosic polymers available under the trade name UCARE® Polymers from Amerchol Corp., Edison, N.J., such as for example but not limited to Polymer JR™. Generally, these cationic cellulose polymers contain quaternized N,N-dimethylamino groups along the cellulosic polymer chain.

Another suitable class of wetting agents for eye-drop solutions are non-polymeric wetting agents. Examples may include glycerin, propylene glycol, and other non-polymeric diols and glycols. The specific quantities of wetting agents used in the invention will vary depending upon the application. However, the wetting agents will typically be included in an amount from about 0.01 to about 5 weight percent, preferably from about 0.1 to about 2 weight percent.

It will be understood that some constituents possess more than one functional attribute. For example, cellulose derivatives are suitable polymeric wetting agents, but are also referred to as “viscosity increasing agents” to increase viscosity of the composition if desired. Glycerin is a suitable non-polymeric wetting agent but is also may contribute to adjusting tonicity.

Eye-drop solutions of the present invention may optionally include one or more sequestering agents to bind metal ions, which in the case of ophthalmic solutions, might otherwise react with protein deposits and collect on contact lenses. Suitable sequestering agents include, for example, but are not limited to ethylenediaminetetraacetic acid (EDTA) and its salts and hydroxyalkylphosphonate (HAP) and its salts. Sequestering agents are preferably present in a minimum of about 0.01 wt. % and/or a maximum of about 0.2 wt. %.

Contact Lenses

Ophthalmic lenses can generally be subdivided into two major classes, namely hydrogel and rigid gas permeable lenses. Non-hydrogels do not absorb appreciable amounts of water, whereas hydrogels can absorb and retain water in an equilibrium state. Hydrogels are widely used as soft contact lens materials. It is known that increasing the hydrophilicity of the contact lens surface improves the wettability of the contact lenses. This in turn is associated with improved wear comfort of contact lenses. Additionally, the surface of the lens can affect the overall susceptibility of the lens to deposition of proteins and lipids from the tear fluid during lens wear. Accumulated deposits can cause eye discomfort or even inflammation.

The aqueous compositions of the invention can be used with all types of contact lenses such as conventional hard, soft and rigid lenses as well as silicone lenses. The term “soft lens” is meant a lens having a proportion of hydrophilic repeat units such that the water content of the lens during use is at least 20% by weight. The term “soft contact lens” as used herein generally refers to those contact lenses that readily flex under small amounts of force. Typically, soft contact lenses are formulated from polymers having a certain proportion of repeat units derived from 2-hydroxyethyl methacrylate and/or other hydrophilic monomers or macromonomers, typically crosslinked with a crosslinking agent. However, newer soft lenses, especially for extended wear, are being made from high-Dk siloxane-containing materials.

Such aqueous compositions can be used to prevent the overgrowth of harmful Gram-negative and Gram-positive bacteria such as Pseudomonas aeruginosa, Serratia marcescens and Staphylococcus aureus, on the lens surfaces during wear, or during the soak time, while being gentle and non-toxic against corneal epithelial cells.

Packaging of Solutions

The eye-drop solutions are typically sold in a wide range of small volume containers from 1 to 30 ml in size, preferably 1 ml to 20 ml in size. Such containers can be made from HDPE (high density polyethylene), LDPE (low density polyethylene), polypropylene, poly(ethylene terepthalate) and the like. Flexible bottles having conventional drop dispensing tops are especially suitable for use with the present invention.

### EXAMPLES

### Example 1

### Formulations

Ophthalmic formulations were prepared using ophthalmically acceptable ingredients in the amounts and combinations represented in the Table Below:

| | Formula 1 | Formula 2 | Formula 3 | Formula 4 |
|---|---|---|---|---|
| Ingredient | % w/w | % w/w | % w/w | % w/w |
| Boric acid | 0.85 | 0.85 | 0.85 | 0.85 |
| Sodium chloride | 0.047 | 0.033 | 0.033 | 0.1917 |
| Sodium phosphate | 0.15 | 0.15 | 0.15 | 0.15 |
| (monobasic) | | | | |
| Sodium phosphate | 0.31 | 0.31 | 0.31 | 0.31 |
| (dibasic) | | | | |
| HAP (30%) | 0.1 | 0.1 | 0.1 | 0.1 |
| Tetronic 1107 | 1.5 | — | — | 1 |
| Pluronic F-127 | 3 | 4.5 | 4.5 | 2 |
| Pluronic P123 | 0.1 | 0.1 | — | — |
| Pluronic P105 | — | — | 0.1 | — |
| Polymer JR | 0.02 | 0.02 | 0.02 | 0.02 |
| Alexidine 2HCl | 3.00 ppm | 3.00 ppm | 3.00 ppm | 4.5 ppm |
| Purified water | Q.S. to | Q.S. to | Q.S. to | Q.S. to |
| | 100% w/w | 100% w/w | 100% w/w | 100% w/w |
| Ph @ 25° C. | 6.8-7.2 | 6.8-7.2 | 6.8-7.2 | 6.8-7.2 |
| Osmolality | 240-280 | 240-280 | 240-280 | 270-300 |
| | mOsm/kg | mOsm/kg | mOsm/kg | mOsm/kg |

Formulae 1, 2, 3 and 4 have been shown to have effective cleaning properties. With Alexidine, it has been discovered that approximately 1 ppm is required to disinfect each of the solutions above. Alexidine present at 4.5 ppm had sufficient antimicrobial activity to disinfect a contact lens when removed from the eye and treated with solution according to one or more disinfecting/cleaning regimens that are known in the art. Three parts per million of Alexidine will prevent growth of microbes on the tissue in the eye or on a contact lens worn in the eye. Likewise, three parts per million will prevent bacterial attachment to the tissue of the eye or to a contact lens worn in the eye. Regular use of each of the drops above will have improved lipid cleaning properties and will manage bacteria or other microbes of a contact lens wearer while in the eye of a patient.

### Example 2

### Bioburden Reduction

Suspensions of challenge bacteria were prepared to the concentration of approximately 1×10⁵ Colony Forming Unit (CFU)/ml in either PBS or in a 100% mixture with organic soil:

Staphylococcus aureus (ATCC 6538)

Pseudomonas aeruginosa (ATCC 9027)

Serratia marcescens (ATCC 13880)

The organic soil, which attempts to mimic natural soiling that could occur in lens cases and on the lens during use, was composed of heat-killed Saccharomyces cerevisiae combined with heat-inactivated fetal bovine serum, which attempts to mimic the natural soiling of lens cases and solutions during their use. This “organic soil” was used to give final concentrations of 1×10⁴/ml heat-killed S. cerevisiae and 0.1% heat inactivated fetal bovine serum in the assays.

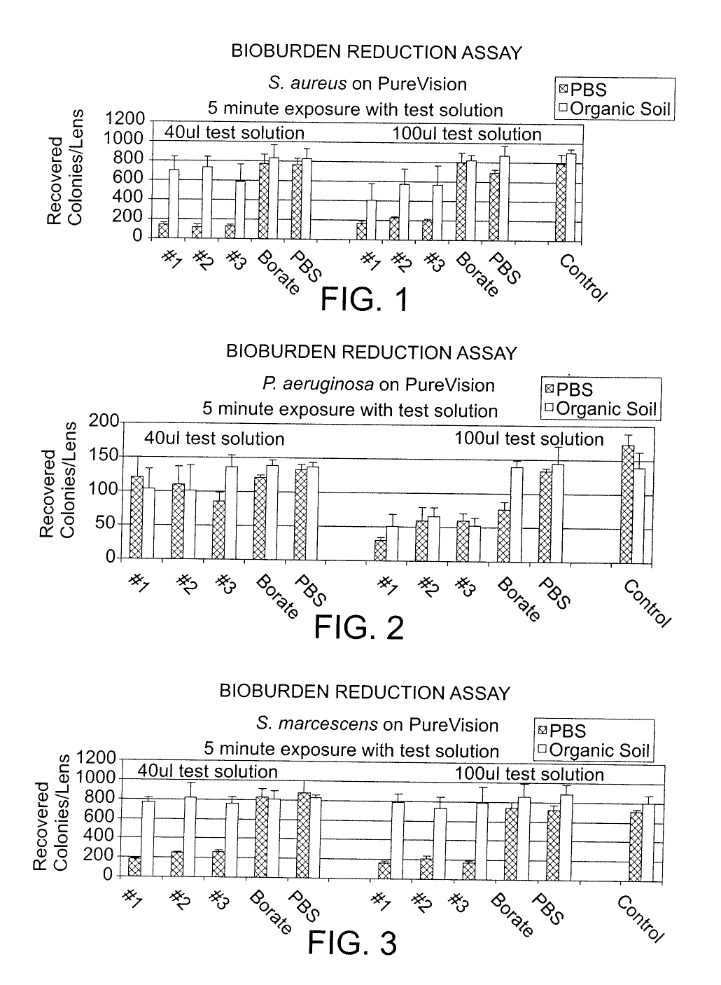

Samples of Purevision® contact lenses were inoculated on the convex side with 10 μl suspension (about 1×10³ CFU) and kept in the room temperature for 10 minutes. Either 40 μl or 100 μl each of Formulations 1-3 solution (as well as comparative solutions of borate) were added to the contact lens and bacteria were exposed for 5 minutes. Exposed contact lenses were then transferred to 3 ml Dey-Engly Neutralizing Broth (DEB). Lenses and DEB dilutions were plated on Tryptic Soy Agar (TSA) growth media. Bacterial recovery was determined after two days. The results are displayed in FIG. 1, FIG. 2 and FIG. 3

What is claimed is:

1. A method of treating contact lenses comprising:
administering to the lens an ophthalmically safe composition comprising an amount of antimicrobial agent that prevents growth of microbes in the eye but is a less than the minimum disinfecting amount.

2. The method of claim 1, wherein the amount of antimicrobial agent is greater than the minimum preserving amount by a multiple of 1.5.

3. The method of claim 1, wherein the antimicrobial agent is a quaternium containing antimicrobial agent.

4. The method of claim 1, wherein the antimicrobial agent is a biguanide containing antimicrobial agent.

5. The method of claim 1, wherein the antimicrobial agent is selected from the group consisting of poly(hexamethylene biguanide), chlorhexadine and Alexidine.

6. The method of claim 1, wherein the antimicrobial agent is Alexidine.

7. The method of claim 1, wherein the surfactant in the composition is selected from the group consisting of poly(ethylene oxide)-poly(propylene oxide) block copolymers and combinations thereof.

8. The method of claim 1, wherein the amount of surfactant in the composition is a minimum of about 0.5 wt. % and a maximum of about 10 wt %.

9. The method of claim 1, wherein the composition further comprises a cationic polysaccharide.

10. The method of claim 1, wherein the amount of cationic polysaccharides in the composition is a minimum of about 0.001 wt. % and a maximum of about 0.5 wt. %.

11. A method of treating contact lenses comprising:
administering to the lens an ophthalmically safe composition comprising an antimicrobial agent in an amount that prevents attachment of bacteria in the eye but is a less than disinfecting amount.

12. The method of claim 11, wherein the amount of antimicrobial agent is greater than the minimum preserving amount by a multiple of 1.5.

13. The method of claim 11, wherein the antimicrobial agent is a quaternium containing antimicrobial agent.

14. The method of claim 11, wherein the antimicrobial agent is a biguanide containing antimicrobial agent.

15. The method of claim 11, wherein the antimicrobial agent is selected from the group consisting of poly(hexamethylene biguanide) chlorhexadine and Alexidine.

16. The method of claim 11, wherein the antimicrobial agent is Alexidine.

17. The method of claim 11, wherein the surfactant in the composition is selected from the group consisting of poly(ethylene oxide)-poly(propylene oxide) block copolymers and combinations thereof.

18. The method of claim 11, wherein the amount of surfactant in the composition is a minimum of about 0.5 wt. % and a maximum of about 10 wt %.

19. The method of claim 11, wherein the composition further comprises a cationic polysaccharide.

20. The method of claim 11, wherein the amount of cationic polysaccharides in the composition is a minimum of about 0.001 wt. % and a maximum of about 0.5 wt. %.

21. A method of treating contact lenses comprising:
administering to the lens an ophthalmically safe composition comprising an antimicrobial agent in an amount that prevents growth of microbes in the eye but is a less than the minimum disinfecting amount.

22. The method of claim 21, wherein the amount of antimicrobial agent is greater than the minimum preserving amount by a multiple of 1.5.

23. The method of claim 21, wherein the antimicrobial agent is a quaternium containing antimicrobial agent.

24. The method of claim 21, wherein the antimicrobial agent is a biguanide containing antimicrobial agent.

25. The method of claim 21, wherein the antimicrobial agent is selected from the group consisting of poly(hexamethylene biguanide), chlorhexadine and Alexidine.

26. The method of claim 21, wherein the antimicrobial agent is Alexidine.

27. The method of claim 21, wherein the surfactant in the composition is selected from the group consisting of poly(ethylene oxide)-poly(propylene oxide) block copolymers and combinations thereof.

28. The method of claim 21, wherein the amount of surfactant in the composition is a minimum of about 0.1 wt. % and a maximum of about 10 wt %.

29. The method of claim 21, wherein the composition further comprises a cationic polysaccharide.

30. The method of claim 21, wherein the amount of cationic polysaccharides in the composition is a minimum of about 0.001 wt. % and a maximum of about 0.5 wt. %.
